(19) United States
(12) Patent Application Publication
(10) Pub. No.: US 2010/0133118 A1
(43) Pub. Date: Jun. 3, 2010

(54) ELECTROCHEMICAL METHODS OF DETECTING NUCLEIC ACID HYBRIDIZATION

(75) Inventors: Ronald G. Sosnowski, Coronado, CA (US); Gabriel Baru Fassio, Burnaby (CA); Zuxu Yao, San Diego, CA (US); Robert Haigis, San Diego, CA (US); Tao Ye, San Diego, CA (US)

Correspondence Address: DLA PIPER LLP (US), 4365 EXECUTIVE DRIVE, SUITE 1100, SAN DIEGO, CA, 92121-2133, US

(73) Assignee: ADNAVANCE TECHNOLOGIES, INC., San Diego, CA (US)

(21) Appl. No.: 12/624,321

(22) Filed: Nov. 23, 2009

Related U.S. Application Data
(60) Provisional application No. 61/117,528, filed on Nov. 24, 2008.

(51) Int. Cl. C12Q 1/68; G01N 27/26

(52) U.S. Cl. 205/777.5; 204/403.01; 205/780.5

(57) ABSTRACT

In accordance with the present invention, there are provided systems for detecting hybridization of nucleic acids using electrochemical methods having improved sensitivity. Such systems include an electrode having a variably charged oligonucleotide probe and a redox probe. In some embodiments, the systems may further include a binding nexus having an immobilized reporter oligonucleotide probe, which hybridizes to a target nucleic acid sequence. The reporter oligonucleotide probe may be naturally charged, uncharged, or either partially negatively or positively charged. Further provided are methods for detecting the presence of a nucleic acid sequence of interest in a sample.

### RELATED APPLICATIONS

This application claims priority under 35 U.S.C. §119(e) of U.S. Provisional application Ser. No. 61/117,528, filed Nov. 24, 2008 which is hereby incorporated by reference in its entirety.

### FIELD OF THE INVENTION

The present invention relates generally to electrochemical methods of detecting nucleic acid hybridization, and more specifically to methods of detecting hybridization by measuring changes in impedance.

### BACKGROUND INFORMATION

DNA hybridization assays are used routinely in genomic analysis, gene expression studies, and, diagnostic assays. The most widely used detection methods rely on labeling of target DNA, usually by fluorescent dyes. Recently, electrochemical techniques for detection of DNA hybridization have been reported in which hybridization is detected using redox-active metal complexes. Such electrochemical methodologies have been demonstrated to provide sequence-specific detection of DNA that is rapid and label-free.

Rates of guanine oxidation catalyzed by electrochemically oxidized transition-metal complexes have been used to evaluate the solvent accessibility of bases for the detection of mismatches in solution. Electrochemical signals triggered by the association of small molecules with DNA have also been applied in the design of other novel biosensors. Toward this end, oligonucleotides have been immobilized on electrode surfaces by a variety of linkages for use in hybridization assays. These include thiols on gold, carbodiimide coupling of guanine residues on glassy carbon, and alkane bisphosphonate films on Al³⁺-treated gold.

Recently, electrochemical techniques suited for detecting hybridization and DNA damage events have been reported. Hybridization can be detected by redox-active metal complexes and drugs that associate selectively and reversibly with DNA. For example, methylene blue, epirubicin. and mitoxantrone have been used as redox-active indicators for the electrochemical detection of hybridization. Label-free detection of hybridization by using the electrochemical signal of guanine has been studied in detail, because guanine is the most redox active nitrogenous base in nucleic acid.

### SUMMARY OF THE INVENTION

In accordance the present invention, there are provided systems for detecting hybridization of nucleic acids using electrochemical methods having improved sensitivity. Such systems include an electrode having a variably charged oligonucleotide probe and a redox probe. In some embodiments, the systems may further include a binding nexus or particle having immobilized oligonucleotide probes attached. The purpose of the binding nexus is to amplify a charge effect associated with the target hybridized at the electrode. The specificity of this effect is provided by the oligonucleotide sequence immobilized to the binding nexus. The charge effect may be a result of the charge of the oligonucleotide or of charge directly associated with the binding nexus. In these embodiments, the oligonucleotide probe immobilized on the binding nexus is designed to hybridize to a first region of a target nucleic acid molecule and the oligonucleotide probe immobilized on the electrode is designed to hybridize to a second region of the target nucleic acid molecule.

In another embodiment of the invention, there are provided methods for detecting hybridization of nucleic acids. Such methods include contacting an electrode having an uncharged or slightly charged oligonucleotide probe with a solution containing a target nucleic acid and a redox probe; and detecting a change in impedance or current generated by electrostatic repulsion or attraction of the redox probe from the electrode, when the target nucleic acid hybridizes to the probe.

In still another embodiment, there are provided methods for detecting the presence of a nucleic acid sequence of interest in a sample. Such methods include contacting an electrode having an uncharged or slightly charged oligonucleotide probe, wherein the probe contains a nucleotide sequence that is complementary to a target nucleic acid sequence of interest, with a sample containing nucleic acids; allowing hybridization to occur between the probe and nucleic acids of the sample containing nucleic acids; further contacting the electrode with a redox probe and detecting a change in impedance or current generated by electrostatic repulsion or attraction of the redox probe relative to the electrode, when the capture oligo hybridizes with a nucleic acid comprising the sequence of interest, thereby identifying the presence of the nucleic acid sequence of interest.

In yet another embodiment, there are provided kits for conducting an assay. Such kits include an electrode having an uncharged or slightly charged oligonucleotide probe attached thereto, and an appropriate redox probe. The oligonucleotide probe is designed to hybridize to a target nucleic acid molecule of interest. The kit may further contain a binding nexus containing an oligonucleotide probe that hybridizes to a second region of the target nucleic acid molecule, and the binding nexus with the oligonucleotide capable of affecting the charge of the surface of the electrode.

### BRIEF DESCRIPTION OF THE INVENTION

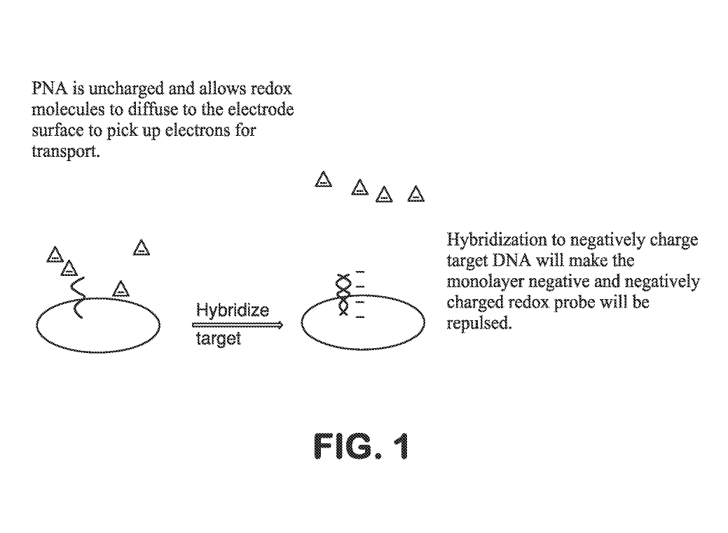

FIG. 1 shows a schematic diagram of a system of the invention with a negative redox probe.

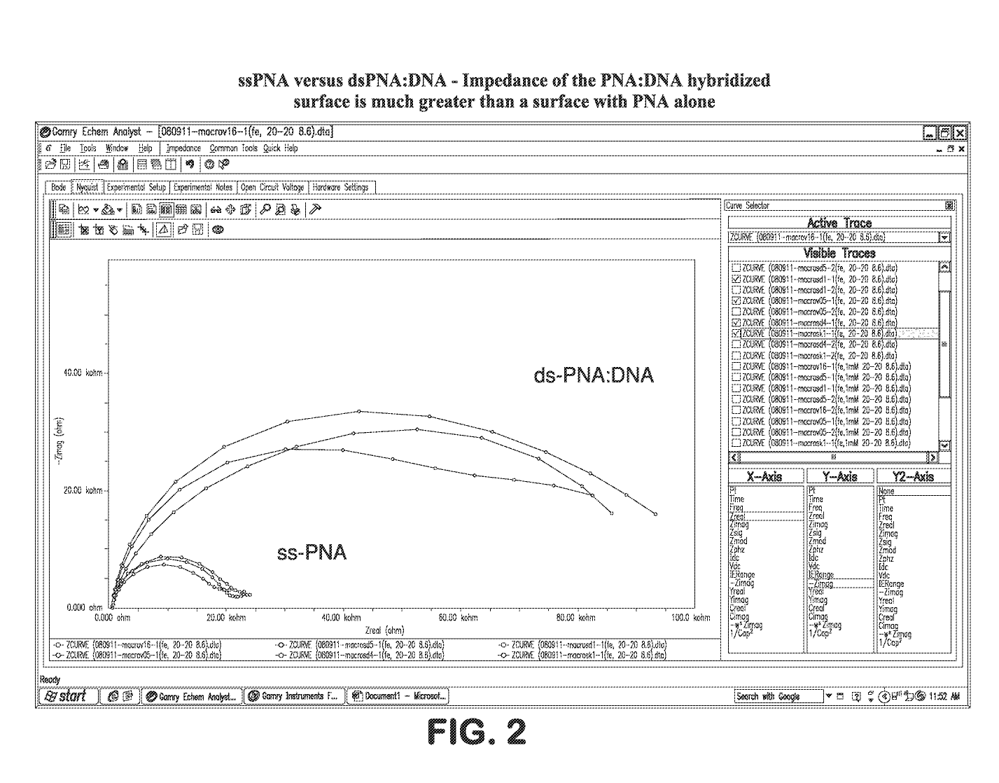

FIG. 2 shows a graph with ssPNA versus dsPNA:DNA.

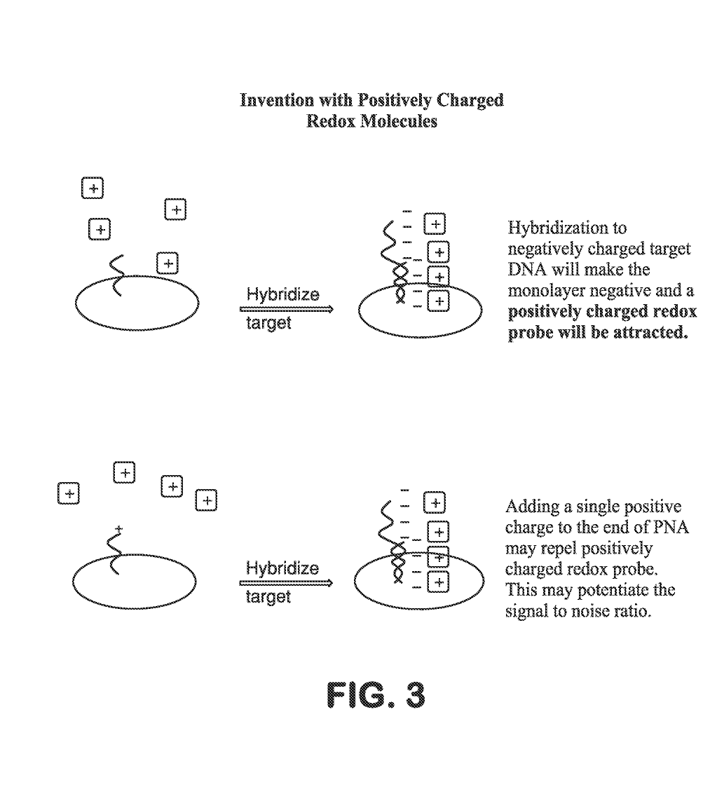

FIG. 3 shows a schematic diagram of a system of the invention with a positive redox probe.

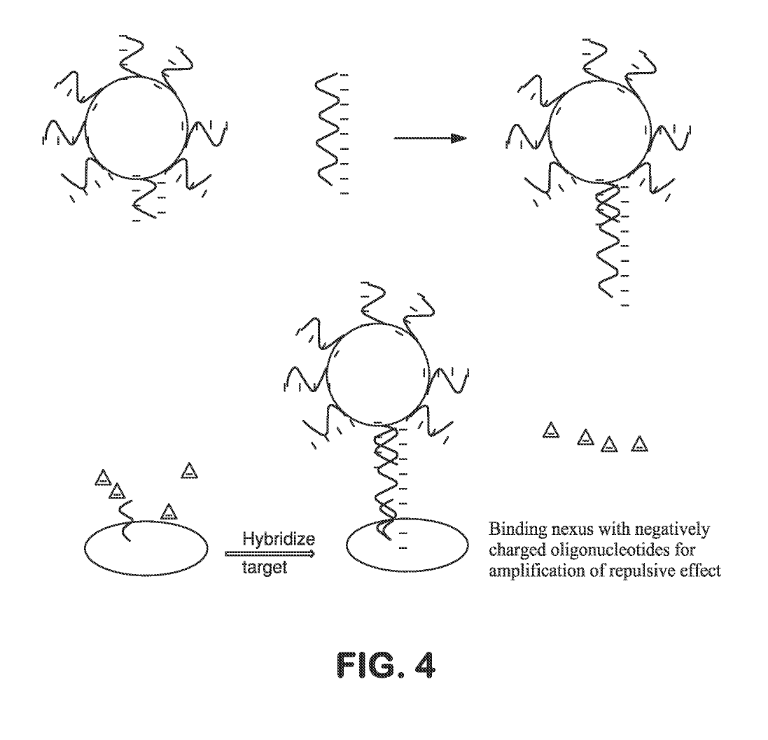

FIG. 4 shows a schematic diagram of a system of the invention.

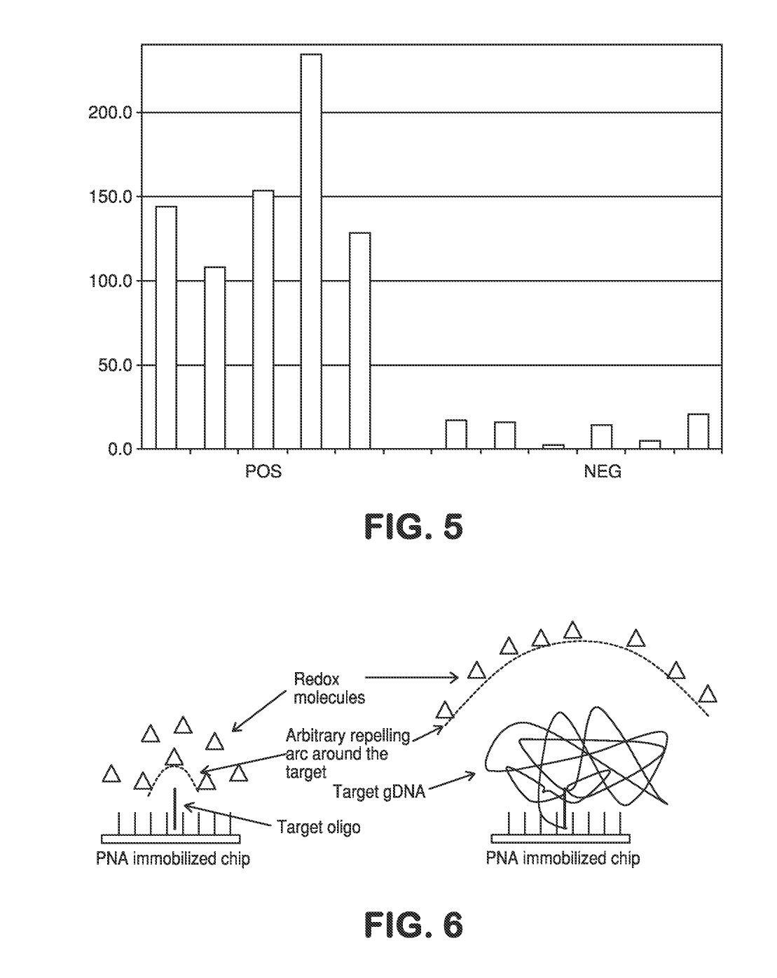

FIG. 5 shows a graph with data from MRSA specific oligonucleotide probes using the methods of the invention.

FIG. 6 shows a comparison between a short oligonucleotide that has hybridized and a long genomic strand of target.

### DETAILED DESCRIPTION OF THE INVENTION

The present invention is based on the discovery that the hybridization of nucleic acid molecules to variably charged oligonucleotides of a self-assembled monolayer (SAM) on the surface of an electrode, can modulate the charge of the monolayer. This change in the charge of the monolayer, and therefore the hybridization of nucleic acid molecules, can be detected by the changes in current or impedance produced by attraction or repulsion of a redox probe. As the redox probe acts to transfer electrons between electrode in an electrochemical cell, the redistribution of redox probe solution density acts to modulate the electrical characteristics of the cell. As provided herein, such systems may be used in, for example, methods of detecting nucleic acid hybridization or methods for detecting the presence of a target nucleic acid sequence of interest in a nucleic acid-containing sample.

One example of an electrochemical technique to determine the presence of target DNA hybridized to the variably charged capture oligonucleotide is electrochemical impedance spectroscopy (EIS). This highly sensitive method is capable of detecting impedances in the gigaohm range. Therefore subtle changes in the electrochemical cell caused by DNA hybridization may be detected and when compared to an equivalent cell that does not have hybridized target, the presence of target sequence in the sample can be determined. Another example of an electrical technique to determine the presence of hybridized DNA is cyclic voltammetry (CV). CV analysis of an electrochemical cell can determine current changes in the nanoamp range.

These and other electrochemical techniques may be used to determine the presence of a target DNA in sample. The optimal method may depend on several factors including the dimension of the electrodes, the type(s) and concentration(s) of the redox probes, the solution components of the cell and other parameters one skilled in the art would recognize to be influential.

In accordance with the present invention, there are provided systems containing an electrode, having an uncharged oligonucleotide probe immobilized thereon, and a redox probe. In some embodiments, the uncharged oligonucleotide probe is designed to hybridize to a target nucleic acid of interest.

As used herein, the term “variably charged oligonucleotide probe” refers to a nucleic acid oligomer or analog thereof which carries a net charge that is different from natural DNA and wherein the variably charged oligonucleotide probe is capable of hybridization to DNA or RNA molecules. Probes may contain about 4 to 100 monomer units, or about 10-50 monomer units, or about 15 to 30 monomer units. In some embodiments, the variably charged oligonucleotide probe is a peptide nucleic acid (PNA). In other embodiments, the uncharged nucleic acid probes are constructed from nucleotide analogs known in the art such as methylphosphonates or phosphotriesters. In certain embodiments, the variably charged oligonucleotide probe may be modified to contain at least one positive or negative charge. In certain embodiment aspects, the oligonucleotide probe may be constructed such that it contains one or more charged nucleotides in combination with uncharged nucleotide analogs. In one aspect, the uncharged oligonucleotide probe is modified to contain a single positive or negative charge. In a particular embodiment, a variably charged oligonucleotide probe will not contribute, to the attraction of a redox probe compared to a probe made of natural DNA. In a preferred embodiment, the variably charged oligonucleotide will affect the redox probe in a manner opposite to the effect of the natural DNA target of interest. In this aspect, the electrical characteristics of the cell with unhybridized variably charged oligonucleotides may be maximally differentiated from the electrical characteristics of the cell with target DNA hybridized to the variably charged oligonucleotides. The target nucleic acid will typically contain a natural phosphate backbone having negatively charged groups which attract positively charged redox probes or repel negatively charged redox probes, thus allowing detection of the hybridized target nucleic acid.

Peptide nucleic acids (PNAs) are polynucleotide mimics, which have a neutral peptide bond providing the backbone between bases. The monomer units of PNAs contain a nucleobase, which allows the PNA molecule to hybridize to complementary nucleic acid strands, via Watson-Crick base pairing, with high affinity and specificity. The various purine and pyrimidine nucleobases are linked to the backbone by methylene carbonyl bonds. In some embodiments, PNA includes an achiral polyamide as the backbone. In one aspect, the N-(2-aminoethyl)glycine forms the backbone. In some embodiments, the PNA contains at least one positive or negative charge. In combination with a positively or negatively charged redox probe, the capture probe may attract or repulse the redox probe thereby affecting the electrochemical characteristics of the cell to alter the impedance and or current. Positive charge may be added to a PNA oligonucleotide with the addition of an ionic amino acid such as lysine. Negative charge may be added by the addition of aspartate. Other methods of altering the charge of the PNA oligonucleotide will be known to one practiced in the art of chemistry. and may include addition of amine groups or carboxylic acid groups.

Methylphosphonates are discussed in: U.S. Pat. No. 4,469,863 (Ts'o et al.); Lin et al., “Use of EDTA derivatization to characterize interactions between oligodeoxyribonucleotide methylphosphonates and nucleic acids,” Biochemistry, 1989, Feb. 7; 28(3):1054-61; Vyazovkina et al., “Synthesis of specific diastereomers of a DNA methylphosphonate heptamer, d(CpCpApApApCpA), and stability of base pairing with the normal DNA octamer d(TPGPTPTPTPGPGPC),” Nucleic Acids Res, 1994 Jun. 25; 22(12):2404-9; Le Bec et al., “Stereospecific Grignard-Activated Solid Phase Synthesis of DNA Methylphosphonate Dimers,” J Org Chem, 1996 Jan. 26; 61 (2):510-513; Vyazovkina et al., “Synthesis of specific diastereomers of a DNA methylphosphonate heptamer, d(CpCpApApApCpA), and stability of base pairing with the normal DNA octamer d(TPGPTPTPTPGPGPC),” Nucleic Acids Res, 1994 Jun. 25; 22(12):2404-9; Kibler-Herzog et al., “Duplex stabilities of phosphorothioate, methylphosphonate, and RNA analogs of two DNA 14-mers,” Nucleic Acids Res, 1991 Jun. 11; 19(11):2979-86; Disney et al., “Targeting a Pneumocystis carinii group I intron with methylphosphonate oligonucleotides: backbone charge is not required for binding or reactivity,” Biochemistry, 2000 Jun. 13; 39(23):6991-7000; Ferguson et al., “Application of free-energy decomposition to determine the relative stability of R and S oligodeoxyribonucleotide methylphosphonates,” Antisense Res Dev, 1991 Fall; 1(3):243-54; Thiviyanathan et al., “Structure of hybrid backbone methylphosphonate DNA heteroduplexes: effect of R and S stereochemistry,” Biochemistry, 2002 Jan. 22; 41(3):827-38; Reynolds et al., “Synthesis and thermodynamics of oligonucleotides containing chirally pure R(P) methylphosphonate linkages,” Nucleic Acids Res, 1996 Nov. 15; 24(22):4584-91; Hardwidge et al., “Charge neutralization and DNA bending by the Escherichia coli catabolite activator protein,” Nucleic Acids Res, 2002 May 1; 30(9):1879-85; and Okonogi et al., “Phosphate backbone neutralization increases duplex DNA flexibility: A model for protein binding,” PNAS U.S.A., 2002 Apr. 2; 99(7):4156-60; all of which are hereby incorporated by reference.

Phosphotriesters are discussed in: Sung et al., “Synthesis of the human insulin gene. Part II. Further improvements in the modified phosphotriester method and the synthesis of seventeen deoxyribooligonucleotide fragments constituting human insulin chains B and mini-CDNA,” Nucleic Acids Res, 1979 Dec. 20; 7(8):2199-212; van Boom et al., “Synthesis of oligonucleotides with sequences identical with or analogous to the 3′-end of 16S ribosomal RNA of Escherichia coli: preparation of m-6-2-A-C-C-U-C-C and A-C-C-U-C-m-4-2C via phosphotriester intermediates,” Nucleic Acids Res, 1977 March; 4(3):747-59; and Marcus-Sekura et al., “Comparative inhibition of chloramphenicol acetyltransferase gene expression by antisense oligonucleotide analogues having alkyl phosphotriester, methylphosphonate and phosphorothioate linkages,” Nucleic Acids Res, 1987 Jul. 24; 15(14):5749-63; all of which are hereby expressly incorporated by reference in their entirety.

Electrodes on which the uncharged oligonucleotide probe may be immobilized are known in the art and include those electrodes use for immobilization of nucleic acids. In some embodiments, the electrode is other than a carbon electrode. In certain embodiments, the electrode is a gold electrode.

Uncharged oligonucleotide probes may be immobilized on the surface of the electrode by methods known in the art for nucleic acid immobilization. For example, PNA probes may be immobilized on the electrode by methods known in the art (e.g., Liu et al., Chem. Commun. 23:2969-71, 2005). Moreover, various strategies used for immobilizing DNA molecules on an electrode by specific covalent adsorption utilizing a reaction between the metal surface of an electrode and an anchoring group of the nucleic acid molecules may also be used. One exemplary method employs a PNA molecule having a terminal thiol, molecular linker group, which binds a metal surface via a sulfur-metal bond (Tornow et al., NanoBioTechnology BioInspired Devices and Materials of the Future, Shoseyov and Levy, Eds., pp. 187-214, Humana Press, 2008). The thiol may be present in an amino acid as cysteine.

In some embodiments, an electrode having a layer of variably charged oligonucleotide probe molecules may be further treated by co-adsorption of short alkanol-thiol molecules, particularly mercaptohexanol (MCH). Such MCH co-adsorption can be employed to control the structure of the PNA layers on the surface. The process of co-adsorption removes and replaces the loosely bound nucleic acids, and changes the specifically bound PNA conformation to an upright position, preventing nonspecific interaction of the specifically bound PNA with the metal surface. Further, any remaining areas of uncovered electrode between bound PNA molecules can be passivated electrochemically and physically by co-adsorption of MCH. Agents as MCH may added alone, after oligonucleotide immobilization has taken place.

Redox probes for use in the present systems and methods may be any of those known to those in the art of electrochemical techniques. Redox probes may be positively or negatively charged, either of which may be paired with variably charged oligonucleotide probe in the present systems. Further, redox probes may be paired with an oligonucleotide probe having a single charge, so that the redox probe and oligonucleotide probe have the same or opposite charge. The skilled artisan will recognize how to pair a positively or negatively charged redox probe with an oligonucleotide probe depending on whether attraction or repulsion of the redox probe is desired. Exemplary redox probes are shown in Table 1 below.

TABLE 1
Exemplary Redox Probes

| Category | Examples |
|---|---|
| Iron Compounds | Fe(CN)₆^{-3/-4}/Fe(NH₃)6^{+3/+2}/Fe(phen)₃^{+3/+2} |
| | Fe(bipy)₂^{+3/+2}/Fe(bipy)₃^{+3/+2} |
| Ruthenium | Ru^{+3/+2}, RuO₄^{-1/-2}Ru(CN)₆^{-3/-4}/Ru(NH₃)6^{+3/+2} |
| Compounds | Ru(en)₃^{+3/+2}/Ru(NH₃)₅(Py)^{+3/+2} |
| Iridium Compounds | Ir^{+4/+3}/Ir(Cl)₆^{-2/-3}/Ir(Br)₆^{-2/-3} |
| Osmium | Os(bipy)₂^{+3/+2}/Os(bipy)₃^{+3/+2}/OsCl₆^{-2/-3} |
| Compounds | |
| Cobalt Compounds | Co(NH₃)6^{+3/+2} |
| Tungsten | W(CN)₈^{-3/-4} |
| Compounds | |
| Molybdenum | Mo(CN)₆^{-3/-4} |
| Compounds | |
| Organic compounds | Ferrocene and derivatives of ferrocene, i.e. mono |
| | and di-carboxilic derivatives, hydroxymethyl |
| | ferrocene) |
| | Quinones: p-benzoquinone/Hydroquinone |
| | Phenol |
| | Ferro/Ferri-Cytochrome; a, a3, b, c, c1 |

In some embodiments, the redox probe is a ruthenium (Ru) complex, wherein the Ru complex is not Ru(NH₃)₅R when R is an electron withdrawing ligand. In some embodiments, the electron withdrawing ligand is a heterocyclic moiety, such as a nitrogen-containing heterocycle including substituted or unsubstituted pyridine, pyrimidine, pyridazine, or pyrazine. Other ligands include phosphite derivatives and isonitrile derivatives. In one aspect, the redox probe is Ru(NH₃)₆³⁺. In other embodiments, the redox probe is Fe(CN)₆^{3-/4-}. In still other embodiments, the redox probe is cytochrome c.

In another embodiment of the invention, there are provided methods for detecting hybridization of nucleic acids. Such methods include contacting an electrode having an uncharged oligonucleotide probe, with a solution containing a single stranded nucleic acid and a negatively charged redox probe; and detecting a change in impedance generated by electrostatic repulsion of the redox probe from the electrode, when the single stranded nucleic acid hybridizes to the probe. In other embodiments, the redox probe is positively charged and a change in current generated by the attraction of the redox probe to the hybridized nucleic acid is detected. In some embodiments, the probe contains at least one positive or negative charge. In one aspect, the oligonucleotide probe is a PNA molecule having at least one positive or negative charge. In another aspect, the oligonucleotide probe comprises methylphosphonates.

In some embodiments of the present invention, a target molecule may be assayed by more than one system in series. In one aspect, the method comprises a first step in which a target nucleic acid is contacted with a system comprising an electrode comprising a variably charged oligonucleotide probe having a single positive or negative charge and a redox probe having the same charge. In a second step the target nucleic acid molecule is with a system comprising an electrode comprising a probe having a single positive or negative charge and a redox probe having the opposite charge. The skilled artisan will recognize that the steps could also be performed in the reverse order.

Electrochemical detection techniques include potential step chronoamperometry, DC cyclic voltammetry, and electrochemical impedance spectroscopy (EIS). In certain embodiments EIS is used to detect differences in impedances between electrochemical cells with variably charged oligonucleotides that are unhybridized relative to those cells that contain target DNA hybridized to the variably charged oligos. In EIS, the binding of the target molecule to electrode surface-immobilized probe may be indicated by a shift in the impedance spectrum of the electrode (Katz and Willner, Electroanalysis 15:913-947, 2003).

The impedance of an electrode undergoing heterogeneous electron transfer through a self-assembled monolayer is usually described on the basis of the model developed by Randles (Discuss. Faraday Soc. 1:11-19, 1947). The equivalent electrical circuit model for DNA consists of resistive and capacitance elements. Rₛ is the solution resistance, Rₓ is the resistance through the DNA, R_{ct} is the charge transfer resistance, C is the double-layer capacitance, and W is the Warburg impedance due to mass transfer to the electrode.

In some embodiments, a conventional three-electrode cell may be used in EIS. Such cells may be enclosed in a grounded Faraday cage. Impedance spectroscopy may be measured with a 1025 frequency response analyzer interfaced to an EG&G 283 potentiostat/galvanostat via GPIB on a PC running Power Suite (Princeton Applied Research). Impedance may be measured at the potential of 250 mV versus Ag/AgCl, and be superimposed on a sinusoidal potential modulation of ±5 mV. The frequencies used for impedance measurements can range from 100 kHz to 100 mHz. The impedance data may be analyzed using the ZSimpWin software (Princeton Applied Research). In certain embodiments, impedance data are plotted as a Nyquist plot (i.e., the imaginary impedance (Z″) versus the real impedance (Z′), recorded as a function of the applied frequency). R_{ct} can be determined by fitting the Nyquist plot using the normal Randles equivalent circuit (Patolsky et al., J Am Chem Soc 123:5194, 2001). By plotting the Rct values versus the corresponding reaction time, the association and dissociation kinetics of the fully matched DNA/PNA duplex can be obtained.

In another embodiment, the sensors of the present invention may be used in methods for detecting single nucleotide polymorphisms in target nucleic acid molecules. Such methods involve varying the hybridization conditions (e.g., hybridization temperature, ionic strength, pH, or components of the buffer used in hybridization or washing) under which a test target nucleic acid (i.e., a target nucleic acid molecule whose polymorphism status is unknown) is allowed to hybridize to the probe on the surface of the electrode. The association or dissociation of the test target nucleic acid can be detected using the systems of the invention. The association or dissociation kinetic parameters (e.g., association or dissociation constants) can be compared to the kinetic parameters of a target nucleic acid molecule that is fully complementary to the probe, as well as to a target sequence having a single mismatch to identify a mismatch in the test target nucleic acid molecule.

In still another embodiment, there are provided methods for detecting the presence of a nucleic acid sequence of interest in a sample. Such methods include contacting an electrode having a variably charged oligonucleotide, wherein the oligo contains a nucleotide sequence that is complementary to a nucleic acid sequence of interest, with a sample containing nucleic acids; allowing hybridization to occur between the variably charged oligo and nucleic acids of the sample containing nucleic acids should the complement be present; further contacting the SAM exposed to the sample with negatively charged oligo and detecting a change in electrochemical characteristics generated by electrostatic repulsion of the redox probe from the electrode, when the probe hybridizes to a nucleic acid comprising the sequence of interest, thereby identifying the presence of the nucleic acid sequence of interest. In other embodiments, the redox probe is positively charged and a change in current generated by the attraction of the redox probe to the hybridized nucleic acid is detected. In some embodiments, the variably charged oligo contains at least one positive or negative charge. In one aspect, the oligonucleotide probe is a PNA molecule having at least one positive or negative charge.

In one aspect of the above embodiment of the invention, the method for detecting the presence of a nucleic acid sequence of interest in a sample includes contacting an electrode having a peptide nucleic acid (PNA) probe, wherein the PNA probe contains a nucleotide sequence that is complementary to a nucleic acid sequence of interest, and wherein further the probe contains at least one positive charge, with a sample containing nucleic acids. Hybridization is allowed to occur between the probe and nucleic acids of the sample containing nucleic acids. The electrode is further contacted with a redox probe having a negative charge and a change in impedance generated by electrostatic repulsion of the redox probe from the electrode is detected when the probe hybridizes to a nucleic acid comprising the sequence of interest, thereby identifying the presence of the nucleic acid sequence of interest.

The target nucleic acid sequence of interest can be essentially any nucleic acid sequence. In some embodiments, the nucleic acid sequence of interest is a sequence associated with a particular disease. In one aspect, the sequence of interest comprises a mutation. In certain embodiments, the sequence of interest is associated with a cell proliferative disorder or cancer. Accordingly, detection of a sequence associated with a disease or disorder in a sample from a subject can be used in the diagnosis of the disease or disorder. In other embodiments, the nucleic acid sequence of interest is from a pathogen. Accordingly, the detection of a sequence from a pathogen can be used in the diagnosis of an infection. Pathogens may be a bacterium, a yeast, a fungus, a parasite, or a virus. In particular embodiments, the pathogen is a bacterium. In one aspect, the bacterium is methicillin-resistant Staphylococcus aureus (MRSA).

In still other embodiments, the systems or methods of the invention further include a binding nexus having immobilized oligonucleotide probes. In these embodiments, the oligonucleotide probe immobilized on the binding nexus is designed to hybridize to a first region of a target nucleic acid molecule and the electrode used in the system or method comprises a variably charged oligonucleotide probe designed to hybridize to a second region of the target nucleic acid molecule. The skilled artisan will recognize that the probes should be designed so that each probe is able to bind to the target nucleic acid molecule simultaneously, without the binding of one probe interfering with the binding of the other. Thus, the binding nexus and electrode are used together in essentially a sandwich format. While not wishing to be bound to any particular theory, it is believed that the use of a binding nexus, to which a multiplicity of target molecules may bind simultaneously, increases the charged nucleic acid molecules at the surface of the electrode, and thereby increases the signal generated by the hybridization of a target molecule (simultaneously hybridized to a binding nexus) to the electrode.

In related embodiments, the format described above may be used in methods of detecting a target nucleic acid molecule in a sample. In this method, the binding nexus acts to capture the target nucleic acid molecule on the surface of the bead via hybridization to a first oligonucleotide probe contained on the surface of the bead. The binding nexuses having the target nucleic acid bound thereto may then be separated from the biological sample by methods known the those of skill in the art. Washing steps may further be incorporated. The presence of the target nucleic acid on the bead may then be detected upon hybridization to a second oligonucleotide probe on the surface of the electrode. In certain embodiments, the bead is a magnetic bead and a magnetic field may be applied to facilitate separation of the bead from the sample. A novel advantage of this method is that the target sequence does not need to be eluted from the binding nexus in order to be analyzed. This saves a step in sample preparation thereby increasing the value of the invention.

In some embodiments, the amplifying repulsive effect of the binding nexus attachment to the target immobilized on the electrode surface may be further enhanced. In one embodiment, a target is first hybridized to variably charged oligonucleotides immobilized on the electrode. A reporter oligonucleotide containing sequence complementary to a second region of the target and containing a biotin moiety is then contacted with the electrode. In the presence of target immobilized on the electrode, the reporter hybridizes with the target. A binding nexus having a biotin receptor bound to it is then contacted with the electrode. The biotin receptor may be for example streptavidin and the binding nexus itself may be a streptavidin molecule. The binding nexus is placed in contact with the nucleic acid complex immobilized on the surface of the electrode. After washing away unbound binding nexus entities, the electrode is contacted with a primary biotinylated amplifying oligonucleotide that has no sequence complementarity to any of the previously incorporated oligonucleotides. Therefore, the amplifying oligonucleotide will only bind to biotin receptor sites on the immobilized binding nexus. This system shall further contain an amplifying target oligonucleotide with a first region complementary to the primary biotinylated amplifying oligonucleotide. The amplifying target sequence shall contain a second region that is complementary to a secondary biotinylated amplifying oligo. A secondary biotinylated oligo is further contacted to the immobilized nucleic acid complex on the electrode. In this way a self-assembling charge amplification network or complex is formed. The self-assembling charge amplification network or complex is a composition including an electrode having a source of electrons, a variably charged oligonucleotide immobilized on the electrode, target DNA hybridized to the variably charged oligonucleotide through a first nucleotide sequence, biotinylated reporter oligo hybridized to a second nucleotide sequence, binding nexus containing a biotin receptor bound to the biotinylated reporter oligo, primary biotinylated amplifying oligo bound to the binding nexus, amplifying target oligo hybridized to the primary biotinylated amplifying oligo through a first amplifying target oligo sequence, secondary amplifying oligonucleotide hybridized to a second amplifying target oligo sequence.

Samples which may be assayed by the invention methods include any sample containing nucleic acid. In some embodiments, the sample is a biological sample. Such samples include but are not limited to any bodily fluid, such as a serum, urine, saliva, plasma, blood, cerebrospinal fluid, tears, pleural fluid, ascites fluid, sputum, stool, pancreatic juice, bile duodenal juice, and any bodily fluid that drains a body cavity or organ. Further examples include cell-containing samples, tissue samples or biopsy samples. Samples may be treated prior to use in the invention methods with a reagent effective for lysing the cells contained in the fluids, tissues, or animal cell membranes of the sample, and for exposing the nucleic acid(s) contained therein. Methods for purifying or partially purifying nucleic acid from a sample may also be employed and are well known in the art (e.g., Sambrook et al., Molecular Cloning: a Laboratory Manual, Cold Spring Harbor Press, 1989, herein incorporated by reference).

The skilled artisan will recognize that the binding nexus used in these embodiments can take many forms, but require that an oligonucleotide probe is able to be immobilized thereon. Examples include, but are not limited, to magnetic beads, agarose beads, polymer beads, microparticles, nanoparticles, proteins with a positive or negative charge, brush DNA, avidin, streptavidin, nuetravidin or combinations thereof. In certain embodiments an avidin, streptavidin, or nuetravidin molecule comprises immobilized charged oligonucleotide probes. Biotinylated probe molecules may be attached to the avidin, streptavidin, or nuetravidin molecule via the avidin-biotin interaction. In these embodiments, the oligonucleotide probe immobilized on the avidin, streptavidin, or nuetravidin molecule is designed to hybridize to a first region of a target nucleic acid molecule and the oligonucleotide probe immobilized on the electrode is designed to hybridize to a second region of the target nucleic acid molecule.

In an alternative embodiment, the binding nexus may itself carry a repulsive or attractive charge relative to the redox molecule. For example, but not to be considered in limitation, a polystyrene bead having attached both streptavidin and carboxylic acid may be employed, resulting in a negatively charged entity at physiological pH values of solution. Biotinylated reporter oligo may be attached to the charged binding nexus and then reacted with the immobilized target. Alternatively, reporter oligo may first be reacted with the immobilized target and then the charged binding nexus may be put in contact with the immobilized nucleic acid complex. The biotinylated reporter oligonucleotide used may be variably charged or native.

In other embodiments, the systems and methods for detecting nucleic acid hybridization may further comprise the use of metal nanoparticles to amplify the signal generated upon hybridization of the target nucleic acid molecule to the probe on the surface of the electrode. In certain of these embodiments, the target nucleic acid molecule is biotinylated and hybridized to the probe on the surface of the probe. Hybridization can be confirmed by, for example, the change of interfacial charge transfer resistance (R_{ct}), experimented by the redox marker. Streptavidin-coated metal nanoparticles (e.g., gold nanoparticles) are added to the system after hybridization of the target. The addition of streptavidin-nanoparticles, binding to the target due to the strong streptavidin-biotin interaction, leads to a further increment of R_{ct}, thus obtaining significant signal amplification (see e.g., Bonanni et al., Electrochimica Acta 53:4022-9, 2008).

Another embodiment of the present invention is a kit for conducting an assay. Such kits include an electrode having an uncharged or slightly charged oligonucleotide probe attached thereto, and an appropriate redox probe. The oligonucleotide probe is designed to hybridize to a target nucleic acid molecule of interest. In certain embodiments, the uncharged oligonucleotide probe will be modified to contain at least one positive or negative charge. In one aspect, the probe is a PNA molecule carrying a single charge. The kit may further contain a bead or particle containing an uncharged or slightly charged oligonucleotide probe that hybridizes to a second region of the target nucleic acid molecule. Additionally, a kit according to the present invention can include other reagents and/or devices which are useful in preparing or using any biological samples, electrodes, probe sequences, target sequences, liquid media, counterions, or detection apparatus, for various techniques described herein or already known in the art.

### Example 1

### Detection of MRSA in Clinical Samples without Amplification

In one illustrative example of the invention Patient sample DNA was obtained from a pathology lab. A partial sample of the DNA was assayed with the Gene Ohm MRSA assay to determine the presence of MRSA. The remainder was subjected to testing with the present invention. Briefly, DNA from three positive samples and three negative samples were pooled to provide sufficient material to allow multiple tests. The pooled DNAs were then run over magnetic beads decorated with oligonucleotide probes complementary to MRSA specific sequence. DNA was eluted from the beads and the volume was reduced by evaporative centrifugation (Speedivac). The resulting volumes were divided and put onto 5 chips (positives) and 6 chips (negatives). All chips contained capture oligonucleotides complementary to a second MRSA specific sequence. An initial EIS (rct) value was obtained prior to hybridization. After hybridization, the chips were again subjected to EIS. The data shown in FIG. 5 reflect the ratios of post-hybridization to pre-hybridization EIS.

### Example 2

### Use of Long Strands of gDNA to Enhance Target Signal

The present invention detects the amount of charge present on the surface of an electrode. Therefore, longer strands of DNA, with concomitant greater negative charge, will give a greater signal response. The complementary sequence of the uncharged PNA capture probe immobilized on the electrode contains relatively few nucleotides, from about 8 to 20. Therefore a target molecule could hybridize with only a few bases, yet have a very wide range of variable charge and therefore signal output. If the genomic DNA is not intentionally fragmented into small uniformly sized fragments, the targets could be thousands of bases long. FIG. 6 shows a comparison between a short oligonucleotide that has hybridized and a long genomic strand of target. Although both cases show hybridization of only one molecule, the long genomic fragment will give a greater signal. Therefore in one embodiment, it will be advantageous to apply unfragmented or partially fragmented nucleic acid, e.g., genomic DNA, to the chip to achieve enhanced sensitivity and detection of a small number of target molecules.

Although the invention has been described with reference to the above examples entire contents of which are incorporated herein by reference, it will be understood that modifications and variations are encompassed within the spirit and scope of the invention. Accordingly, the invention is limited only by the following claims.

What is claimed is:

1. A system comprising:
an electrode comprising a variably charged oligonucleotide probe; and
a redox probe.

2. The system of claim 1, wherein the variably charged oligonucleotide probe is immobilized to the electrode through chemical bonds such as covalent bonds, hydrogen bonds, electrostatic bonds and/or van der Waals forces.

3. The system of claim 2, wherein the variably charged oligonucleotide probe has a region that is complementary to a first region of the target nucleic acid sequence.

4. The system of claim 1, wherein the variably charged oligonucleotide probe is a peptide nucleic acid (PNA), a methylphosphonate oligomer or a phosphotriester oligomer.

5. The system of claim 3, wherein the probe is PNA and carries no charge.

6. The system of claim 3, wherein the probe is PNA and carries a variable number of positive charges.

7. The system of claim 6, wherein the probe is PNA and wherein the number of positive charges range from about 1 to 10.

8. The system of claim 3, wherein the probe is PNA and carries a variable number of negative charges.

9. The system of claim 8, wherein the number of negative charges range from about 1 to 10.

10. The system of any of claim 4, 6 or 8 wherein the redox probe is negatively charged.

11. The system of any of claim 4, 6 or 8, wherein the redox probe is positively charged.

12. The system of claim 1, wherein the variably charged oligonucleotide probe and the redox probe carry the same net charge.

13. The system of claim 1, wherein the variably charged oligonucleotide probe and the redox probe carry a different net charge.

14. The system of claim 1, wherein the redox probe is a ruthenium (Ru) complex.

15. The system of claim 1, wherein the redox probe is a ferri-ferro cyanide complex.

16. The system of claim 1, wherein the electrode material is selected from the group consisting of gold, carbon and platinum.

17. The system of claim 1, wherein the redox probe is selected from the group consisting of Fe(CN)₆^{-3/-4}, Fe(NH₃)6^{+3/+2}, Fe(phen)₃^{+3/+2}, Fe(bipy)₂^{+3/+2}, Fe(bipy)₃^{+3/+2}, Ru^{+3/+2}, RuO₄^{-1/-2}Ru(CN)₆^{-3/-4}/Ru(NH₃)6^{+3/+2}, Ru(en)₃^{+3/+2}/Ru(NH₃)₅(Py)^{+3/+2}, Ir^{+4/+3}/Ir(Cl)₆^{-2/-3}/Ir(Br)₆^{-2/-3}, Os(bipy)₂^{+3/+2}/Os(bipy)₃^{+3/+2}/OsCl₆^{-2/-3}, Co(NH₃)6^{+3/+2}, W(CN)₈^{-3/-4}, Mo(CN)₆^{-3/-4}, Ferrocene, mono-carboxilic derivatives of ferrocene, di-carboxilic derivatives of ferrocene, hydroxymethyl ferrocene, p-benzoquinone, hydroquinone, phenol, ferro/ferri-cytochrome a, ferro/ferri-cytochrome a3, ferro/ferri-cytochrome b, ferro/ferri-cytochrome c, and ferro/ferri-cytochrome c1.

18. The system of claim 1, further comprising a binding nexus having an immobilized oligonucleotide probe, wherein the probe immobilized on the binding nexus is designed to hybridize to a first region of a target nucleic acid molecule.

19. The system of claim 18, wherein the binding nexus is selected from the group consisting of magnetic beads, agarose beads, polymer beads, polylysine beads, gold beads, microparticles, nanoparticles, proteins with a positive or negative charge, uncharged proteins, brush DNA, avidin, streptavidin, nuetravidin and polysaccharides.

20. The system of claim 18, wherein the binding nexus is networked to a plurality of binding nexuses.

21. The system of claim 20, wherein the linking agent is complementary oligonucleotides.

22. The system of claim 18 wherein the oligonucleotide probe immobilized on the binding nexus is a natural nucleic acid polymer having negative charges.

23. The system of claim 18 wherein the binding nexus carries a variable charge.

24. The system of claim 1, further comprising an active signal amplifying entity, having an immobilized oligonucleotide probe, wherein the probe immobilized on the binding nexus is designed to hybridize to a first region of a target nucleic acid molecule.

25. The system of claim 24, wherein the active signal amplifying entity is an enzyme that catalyzes synthesis of a product that affects electron transfer.

26. The system of claim 25, wherein the enzyme is selected from alkaline phosphatase or a kinase.

27. The system of claim 1, further comprising an electrostatic binding entity to change the net charge of the nucleic acid hybrid.

28. The system of claim 27, wherein the electrostatic binding entity is polyaniline polymerized by addition of horse radish peroxidase.

29. A method for detecting hybridization of nucleic acids, comprising:
contacting an electrode comprising a variably charged oligonucleotide (VCO) probe, with a sample containing a target nucleic acid and a charged redox probe; and
detecting a change in impedance as a result of the target nucleic acid hybridizing to the probe.

30. The method of claim 29, wherein the variably charged oligonucleotide probe is immobilized to the electrode through chemical bonds selected from covalent bonds, hydrogen bonds, electrostatic bonds or van der Waals forces.

31. The method of claim 29, wherein the variably charged oligonucleotide probe has a region that is complementary to a first region of the target nucleic acid sequence.

32. The method of claim 29, wherein the VCO probe is uncharged.

33. The method of claim 29, wherein the VCO probe is modified to contain at least one positive or negative charge.

34. The method of claim 29, wherein the VCO probe is a peptide nucleic acid (PNA), a methylphosphonate oligomer or a phosphotriester oligomer.

35. The method of claim 34, wherein the probe is PNA and carries at least a single charge.

36. The system of claim 29, wherein the net charge of the VCO probe the redox probe are the same.

37. The system of claim 29, wherein the net charge sign of the VCO probe and the redox probe are different.

38. The method of claim 29, wherein the redox probe is a ruthenium (Ru) complex.

39. The method of claim 29, wherein the redox probe is a Ferro-Ferri cyanide complex

40. The system of claim 29, wherein the electrode material is selected from the group consisting of gold, carbon and platinum.

41. The system of claim 29, wherein the redox probe is selected from the group consisting of Fe(CN)₆^{-3/-4}, Fe(NH₃)6^{+3/+2}, Fe(phen)₃^{+3/+2}, Fe(bipy)₂^{+3/+2}, Fe(bipy)₃^{+3/+2}, Ru^{+3/+2}, RuO₄^{-1/-2}Ru(CN)₆^{-3/-4}/Ru(NH₃)6^{+3/+2}, Ru(en)₃^{+3/+2}/Ru(NH₃)₅(Py)^{+3/+2}, Ir^{+4/+3}/Ir(Cl)₆^{-2/-3}/Ir(Br)₆^{-2/-3}, Os(bipy)₂^{+3/+2}/Os(bipy)₃^{+3/+2}/OSCl₆^{-2/-3}, Co(NH₃)6^{+3/+2}, W(CN)₈^{-3/-4}, Mo(CN)₆^{-3/-4}, Ferrocene, mono-carboxilic derivatives of ferrocene, di-carboxilic derivatives of ferrocene, hydroxymethyl ferrocene, p-benzoquinone, hydroquinone, phenol, ferro/ferri-cytochrome a, ferro/ferri-cytochrome a3, ferro/ferri-cytochrome b, ferro/ferri-cytochrome c, and ferro/ferri-cytochrome c1.

42. The method of claim 29, further comprising a binding nexus having an immobilized oligonucleotide probe, wherein the probe immobilized on the particle is designed to hybridize to a first region of a target nucleic acid molecule.

43. The method of claim 29, wherein the binding nexus is selected from the group consisting of magnetic beads, agarose beads, polymer beads, polysine beads, microparticles, nanoparticles, uncharged proteins, proteins with a positive or negative charge, brush DNA, avidin, streptavidin, nuetravidin and polysaccharides.

44. The method of claim 29, further comprising an active signal amplifying entity, having an immobilized oligonucleotide probe, wherein the probe immobilized on the binding nexus is designed to hybridize to a first region of a target nucleic acid molecule.

45. The method of claim 44, wherein the active signal amplifying entity is an enzyme that catalyzes synthesis of a product that affects electron transfer.

46. The method of claim 45, wherein the enzyme is selected from alkaline phosphatase or a kinase.

47. The method of claim 29, further comprising an electrostatic binding entity to change the net charge of the nucleic acid hybrid.

48. The method of claim 47, wherein the electrostatic binding entity is polyaniline polymerized by addition of horse radish peroxidase.

49. A method for detecting the presence of a nucleic acid sequence of interest in a sample, comprising:
contacting an electrode comprising a VCO probe, wherein the VCO probe comprises a nucleotide sequence that is complementary to a nucleic acid sequence of interest, with a sample containing nucleic acids;
allowing hybridization to occur between the VCO probe and nucleic acids of the sample;
contacting the electrode with a redox probe; and
detecting a change in impedance, thereby identifying the presence of the target nucleic acid.

50. The method of claim 49, wherein the variably charged oligonucleotide probe is immobilized to the electrode through chemical bonds including covalent bonds, hydrogen bonds, electrostatic bonds or van der Waals forces.

51. The system of claim 49, wherein the variably charged oligonucleotide probe has a region that is complementary to a first region of the target nucleic acid sequence.

52. The method of claim 49, wherein the VCO probe is uncharged.

53. The method of claim 49, wherein the VCO probe is modified to contain a single positive or negative charge.

54. The method of claim 49, wherein the VCO probe is a peptide nucleic acid (PNA), a methylphosphonate oligomer or a phosphotriester oligomer.

55. The method of claim 51, wherein the probe is PNA and carries at least single charge.

56. The system of claim 49, wherein the VCO probe and the redox probe carry the same net charge.

57. The system of claim 49, wherein the VCO probe and the redox probe carry a different net charge.

58. The method of claim 49, wherein the redox probe is a ruthenium (Ru) complex.

59. The method of claim 20, wherein the electrode is selected from the group comprising gold, carbon, and platinum.

60. The method of claim 49, wherein the redox probe is selected from the group consisting of Fe(CN)₆^{-3/-4}, Fe(NH₃)6^{+3/+2}, Fe(phen)₃^{+3/+2}, Fe(bipy)₂^{+3/+2}, Fe(bipy)₃^{+3/+2}, Ru^{+3/+2}, RuO₄^{-1/-2}Ru(CN)₆^{-3/-4}/Ru(NH₃)6^{+3/+2}, Ru(en)₃^{+3/+2}/Ru(NH₃)₅(Py)^{+3/+2}, Ir^{+4/+3}/Ir(Cl)₆^{-2/-3}/Ir(Br)₆^{-2/-3}, Os(bipy)₂^{+3/+2}/Os(bipy)₃^{+3/+2}/OsCl₆^{-2/-3}, Co(NH₃)6^{+3/+2}, W(CN)₈^{-3/-4}, Mo(CN)₆^{-3/-4}, Ferrocene, mono-carboxilic derivatives of ferrocene, di-carboxilic derivatives of ferrocene, hydroxymethyl ferrocene, p-benzoquinone, hydroquinone, phenol, ferro/ferri-cytochrome a, ferro/ferri-cytochrome a3, ferro/ferri-cytochrome b, ferro/ferri-cytochrome c, and ferro/ferri-cytochrome c1.

61. The method of claim 49, further comprising a binding nexus having an immobilized oligonucleotide probe, wherein the probe immobilized on the binding nexus is designed to hybridize to a first region of a target nucleic acid molecule.

62. The method of claim 57, wherein the binding nexus is selected from the group consisting of magnetic beads, agarose beads, polymer beads, polylysine beads gold beads, microparticles, nanoparticles, uncharged proteins, proteins with a positive or negative charge, brush DNA, avidin, streptavidin, nuetravidin and polysaccharides.

63. The method of claim 49, further comprising an active signal amplifying entity, having an immobilized oligonucleotide probe, wherein the probe immobilized on the binding nexus is designed to hybridize to a first region of a target nucleic acid molecule.

64. The method of claim 63, wherein the active signal amplifying entity is an enzyme that catalyzes synthesis of a product that affects electron transfer.

65. The method of claim 64, wherein the enzyme is selected from alkaline phosphatase or a kinase.

66. The method of claim 49, further comprising an electrostatic binding entity to change the net charge of the nucleic acid hybrid.

67. The method of claim 66, wherein the electrostatic binding entity is polyaniline polymerized by addition of horse radish peroxidase.

68. The method of claim 49, wherein the nucleic acid sequence of interest is associated with a disease or disorder.

69. The method of claim 59 wherein the nucleic acid sequence of interest is associated with a human genetic disease.

70. The method of claim 59, wherein the disease or disorder is cancer.

71. The method of claim 49, wherein the nucleic acid sequence comprises a mutation.

72. The method of claim 49, wherein the nucleic acid sequence of interest is from a pathogen.

73. The method of claim 62, wherein the pathogen is selected from the group consisting of a bacterium, a yeast, a fungus, a parasite, and a virus.

74. The method of claim 63, wherein the pathogen is a bacterium.

75. The method of claim 64, wherein the bacterium is methicillin-resistant Staphylococcus aureus (MRSA).
